(19) United States
(12) Patent Application Publication
(10) Pub. No.: US 2025/0145943 A1
(43) Pub. Date: May 8, 2025

(54) CELL ENRICHMENT

(71) Applicant: Vow Group Pty Ltd, New South Wales (AU)

(72) Inventors: James Ryall, New South Wales (AU); George Peppou, New South Wales (AU); Rebecca Screnci, New South Wales (AU); Michael Medynskyj, New South Wales (AU)

(21) Appl. No.: 18/832,788

(22) PCT Filed: Mar. 2, 2023
(86) PCT No.: PCT/AU2023/050140

§ 371 Date: Jul. 24, 2024

(30) Foreign Application Priority Data
Mar. 3, 2022 (AU) 2022900514

(51) Int. Cl. C12N 5/00; A23L 13/00; C12M 1/32; C12N 5/077

(52) U.S. Cl. CPC C12N 5/0081; A23L 13/00; C12M 23/12; C12N 5/0656; C12N 5/0658; C12N 2509/00; C12N 2533/54

(57) ABSTRACT

The present disclosure relates to methods for producing an enriched cell population from a heterogeneous cell population. The methods may involve selective culturing of a heterogenous to produce a selected cell population, sorting the selected cell population to produce a population of sorted cells and culturing the population of sorted cells to produce an enriched cell population. The present disclosure also provides compositions comprising cells produced by these methods.

### FIELD OF THE DISCLOSURE

The present disclosure relates to methods for producing an enriched cell population from a heterogeneous cell population.

### BACKGROUND OF THE DISCLOSURE

Any discussion of the prior art throughout the specification should in no way be considered as an admission that such prior art is widely known or forms part of the common general knowledge in the field.

Modern agriculture is much more productive now than it was only a decade ago. To keep up with growing demand, global meat production has tripled over the last 50 years. Despite the efficiency gains, however, agriculture still consumes vast quantities of resources and is a major contributor to greenhouse gas emissions, environmental damage, human sickness and animal suffering. The threats posed by agriculture will only become greater as global meat consumption increases with human population growth.

Agriculture has been reported to use more freshwater than any other human activity, with nearly a third required for livestock (Godfray et al., 2018, Science. 361:243). The vast majority of the water used in livestock production—about 98%—is used to produce animal feed (Godfray et al., 2018, Science. 361:243). Several thousand litres of water and approximately 25 kg of grain are required to produce a single kilogram of beef (Smil V, Should We Eat Meat, Wiley-Blackwell, 2013).

According to the Food and Agriculture Organisation of the United Nations, about 18% of all greenhouse gas emissions are a consequence of animal agriculture. Livestock alone accounts for about 5% of the CO₂ that humans add to the atmosphere (Godfray et al., 2018, Science. 361:243). Amplifying the consequence of these emissions is the loss of natural habitats that might otherwise sequester carbon emissions. The majority of tropical deforestation is for the purpose of feeding animals.

Animal agriculture also presents health and safety risks. Livestock can act as a reservoir for pathogens that infect humans, including E. coli, Salmonella and campylobacter. Intensive factory farming is thought to be responsible for foodborne illnesses such as swine influenza and avian influenza. Moreover, the widespread use of antibiotics in animal agriculture can promote the development of antibiotic-resistant pathogens.

Cultured animal cells—sometimes referred to as cultured meat, cultivated meat, clean meat or in vitro meat—present an alternative source of animal protein that may be safer, less environmentally destructive and more humane compared to traditionally farmed meat. Cultured meat also lends itself to new culinary applications with tailored flavour, texture and nutritional profiles.

But to culture animal cells on a commercial scale, and to promote widespread adoption of cultured meat, the processes, materials and equipment used to produce it should be made cost-competitive. A major challenge in cultured meat production is purifying a specific cell type from a heterogenous cell population obtained from an animal. Known methods of purifying human and mouse cells involve labelling cell surface markers followed by immunofluorescence sorting. These methods are limited by the level of purity achievable, which is typically about 90 to 95% of the desired cell type. Further, the methods are typically species-dependant due to differences in cell surface markers and antibody reactivity.

It is an object of the present invention to overcome or ameliorate at least one of the disadvantages of the prior art, or to provide a useful alternative.

### SUMMARY OF THE DISCLOSURE

In work leading to the present disclosure, the inventors developed methods for producing an enriched cell population from a heterogenous cell population. The cell populations are enriched in the sense that they comprise one or more specific cell types at a higher concentration compared to the starting cell population, which may be a heterogenous population obtained from an animal. For example, the methods described herein may provide a 2-fold enrichment of a particular cell type. In some instances, the enriched cell population consists of only 1 or 2 cell types. The methods described herein can be applied to cells from different species of animals. Moreover, the methods do not require antibody-based cell sorting and can be implemented to yield high throughput results.

In a first aspect, the present disclosure provides a method of producing an enriched cell population, the method comprising:
selectively culturing a heterogenous cell population to produce a selected cell population;
sorting the selected cell population to produce a population of sorted cells; and
culturing the population of sorted cells to produce an enriched cell population.

In some examples, the selective culturing comprises culturing cells of the heterogeneous cell population and selecting cells having a defined phenotype. The defined phenotype is preferably adherence.

The selective culturing may comprise culturing cells of the heterogeneous cell population in a vessel that does not comprise an adherence-enhancing substrate and selecting adherent cells.

In some examples, the selective culturing comprises culturing cells of the heterogeneous cell population in a vessel that does not comprise an adherence-enhancing substrate and selecting non-adherent cells. The selective culturing may further comprise subculturing the non-adherent cells in a vessel comprising an adherence-enhancing substrate. The adherence-enhancing substrate may be gelatin.

In some examples, the selective culturing is performed for at least two weeks.

The selective culturing may comprise dissociating cells from a substrate and subculturing in fresh growth medium. In some examples, the selective culturing comprises iteratively dissociating cells from a substrate and subculturing in fresh growth medium two or more times.

The selected cell population is preferably sorted by fluorescence-activated cell sorting (FACS).

In some examples, the selected cell population is sorted (e.g., by FACS) based on a cellular phenotype. The FACS may sort the selected cell population based on cell size. In some examples, the FACS sorts the selected cell population based on cell viability. Preferably, the FACS does not require an antibody to sort the selected cell population.

In some examples, sorted cells having a defined characteristic are selected and subcultured to produce the enriched cell population.

The step of culturing the population of sorted cells may comprise:
selecting sorted cells having a defined characteristic from the population of sorted cells; and
subculturing the sorted cells having a defined characteristic to produce the enriched cell population.

The defined characteristic may be growth rate or abundance.

In some examples, the population of sorted cells is distributed across a plurality of wells of a multi-well plate. The wells may be imaged and sorted cells having a defined characteristic may be selected and subcultured to produce the enriched cell population.

In some examples, the sorted cells having a defined characteristic are iteratively selected and subcultured.

In some examples, the heterogenous cell population is obtained from an animal. The animal may be a mammal, a marsupial, a reptile or a bird.

In some examples, the enriched cell population consists of less than four cell types. In some examples, the enriched cell population consists of one cell type. At least 98% of cells in the enriched cell population may consist of one cell type. The cell type may be a myoblast or a fibroblast.

In another aspect, the present disclosure provides a method of producing an enriched cell population, the method comprising:
sorting a selected cell population to produce a population of sorted cells; and
culturing the population of sorted cells to produce an enriched cell population,
wherein the selected cell population is obtained by selectively culturing a heterogenous cell population to produce a selected cell population.

In another aspect, the present disclosure provides an enriched cell population produced by a method described herein.

In another aspect, the present disclosure provides a food composition comprising an enriched cell population produced by a method described herein.

### BRIEF DESCRIPTION OF THE DRAWINGS

FIG. 1. A method of producing an enriched cell population according to one aspect of the present disclosure. (A) Isolation of a tissue sample to produce a heterogeneous cell population. (B) selective culturing of the heterogenous cell population to produce a selected cell population. (C) Sorting the selected cell population followed by expansion to produce an enriched cell population. (D) Characterisation of the enriched cell population.

FIG. 2. A method of preparing a heterogeneous cell population according to one aspect of the present disclosure. Tissue is obtained from an animal and physically and/or enzymatically digested to produce a mononuclear suspension of cells.

FIG. 3. A method of selective culturing based on adherence according to one aspect of the present disclosure. Cells are separated into adherent (containing fibroblasts) and non-adherent (containing myoblasts) cells. Each cell population is then grown and expanded.

FIG. 4. A method of single cell sorting according to one aspect of the present disclosure. Cells of the selected cell population are sorted (1 cell/well) into 384-well plates. Cell numbers are then continuously expanded until a sufficient number of cells is produced for characterisation.

FIG. 5. Phase contrast images of cells of the heterogeneous cell population and the selected cell population. (A) Processed crocodile muscle biopsy when first seeded into a six-well plate (completion of the method illustrated in FIG. 2). (B) Adherent cells after media exchange. (C) Non-adherent cells after media exchange. (D) Adherent cells after 5 days of expansion.

FIG. 6. Adherent cells isolated from chicken and duck. (A) Leghorn Chicken cells cultured for 7 days. (B) Muscovy Duck cells cultured for 7 days. (C) Khaki Campbell Duck cells cultured for 14 days. (D) Silkie Chicken cells cultured for 5 days.

FIG. 7. Dot plots of unstained cells sorted by FACS. (A) Crocodile, and (B) sheep fibroblasts were sorted based on forward and side-scatter light (FSC and SSC respectively). The x/y axes represent FSC and SSC of the light shone onto the cell. FSC is proportional to the size of the cell, while SSC is proportional to the granularity of the cell. (A) shows a total of 2,086 events, with each event being a measure of FSC and SSC light deviation. Of these events, 1,076 were determined to be cells. (B) shows a total of 311,535 events, of which 157,066 were determined to be cells.

FIG. 8. Dot plots of Hoechst stained and unstained sheep cells sorted by FACS. (A) Hoechst stained cells can be selected by their light emission following excitation with UV. (B) Unstained cells do not respond to UV excitation. (A) shows Hoechst stained cells exhibiting a high excitation signal after shining a UV light at the cells (y-axis is a UV detector). (B) shows cells not stained with Hoechst exhibiting little to no signal on the x-axis.

FIG. 9. Immunofluorescence staining of pure sheep myoblast cells enriched by a method of the present disclosure. Cells are labelled with (A) Dapi (a fluorescent nuclear stain), (B) MyoD (a myoblast-specific marker), and then the resulting images are merged to show that all Dapi positive cells are also MyoD positive (C).

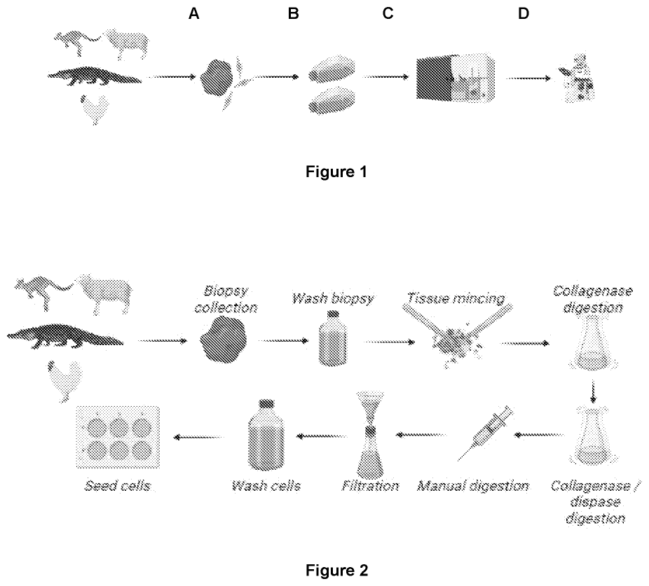

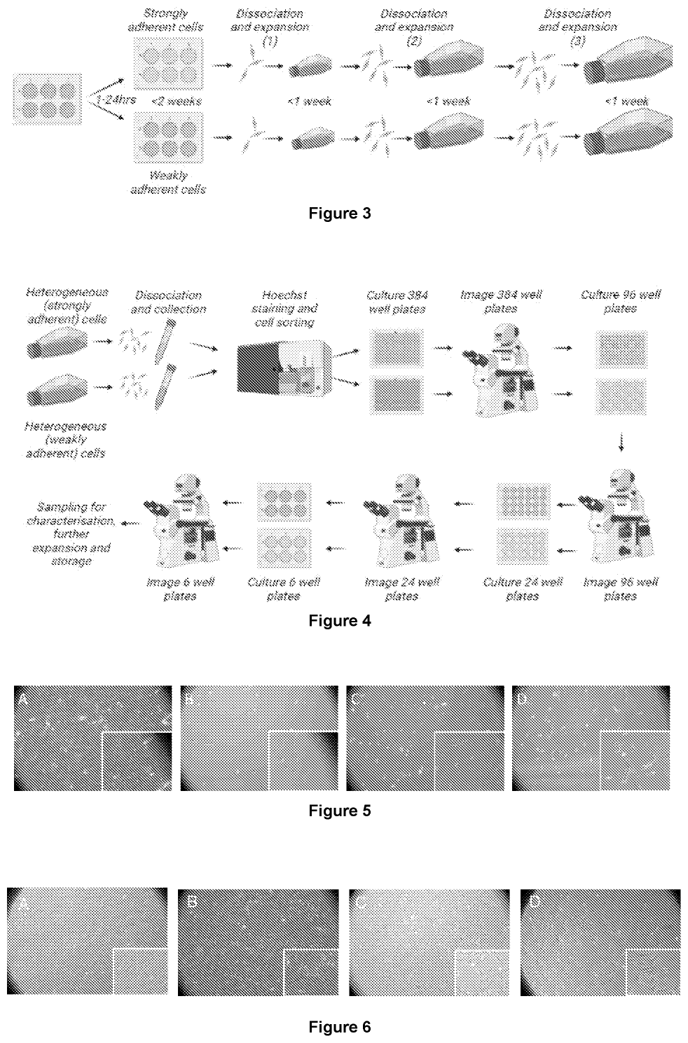

### DETAILED DESCRIPTION

### Definitions

In the context of this specification, the terms “a” and “an” are used herein to refer to one or to more than one (i.e. to at least one) of the grammatical object of the article. By way of example, “an element” means one element or more than one element.

The term “about” is understood to refer to a range of +/−10%, preferably +/−5% or +/−1% or, more preferably, +/−0.1%.

The terms “comprise”, “comprises”, “comprised” or “comprising”, “including” or “having” and the like in the present specification and claims are used in an inclusive sense, ie, to specify the presence of the stated features but not preclude the presence of additional or further features.

The term “isolated” as used herein refers to material that is substantially or essentially free from components that normally accompany it in its native state. For example, an isolated polynucleotide refers to a polynucleotide which has been purified from the sequences which flank it in a naturally-occurring state, eg, a DNA fragment which has been removed from the sequences that are normally adjacent to the fragment.

The term “myotube” refers to a multi-nucleated cell that is formed from the fusion of a plurality of myoblasts and/or myocytes. Myotubes may be identified microscopically as forming thread like cells with multiple nuclei per cell. Nuclei may be visualised by staining. Enhanced expression of Myh3 may also be characteristic of myotubes.

A “progenitor cell” is a cell that is capable of differentiating along one or a plurality of developmental pathways. Some types of progenitor cells may be capable of self-renewal, others may not. A myoblast is a progenitor cell that can differentiate through myogenesis to give rise to a muscle cell, such as a skeletal muscle myocyte or a myotube. The myoblast itself may be derived from a progenitor cell, such as an embryonic stem cell, a pluripotent stem cell or an induced pluripotent stem cell.

The term “serum” refers to a substantially cell-free proteinaceous blood fraction obtained from an animal (eg, fetal bovine serum, horse serum).

The term “stem cell” refers to a subset of progenitor cells that have the capacity or potential, under particular circumstances, to differentiate into a more specialised cell type, and which retains the capacity, under certain circumstances, to proliferate without substantially differentiating. In that regard, stem cells are generally capable of self-renewal.

Where numerical ranges are used to describe certain embodiments of the present disclosure, it will be understood that each range should be considered to encompass subranges therein. For example, the description of a range such as from 1 to 6 should be considered to include subranges such as from 1 to 5, from 2 to 4, from 2 to 6 and so on. Likewise, the description of a range of between 1 and 6 should be considered to include subranges such as between 2 and 5, between 1 and 3, between 3 and 6 and so on.

### Heterogeneous Cell Populations

The methods described herein produce enriched cell populations from heterogeneous cell populations. The heterogenous cell population may comprise more than 2 cell types, such as more than 3 cell types, or more than 4 cell types, or more than 5 cell types, or more than 10 cell types, or more than 15 cell types, or more than 20 cell types, or more than 25 cell types, or more than 30 cell types, or more than 35 cell types, or more than 40 cell types, or more than 45 cell types, or more than 50 cell types, or more than 55 cell types, or more than 60 cell types, or more than 65 cell types, or more than 70 cell types, or more than 75 cell types, or more than 80 cell types, or more than 85 cell types, or more than 90 cell types, or more than 95 cell types, or more than 100 cell types.

For example, the heterogenous cell population may comprise muscle cells, satellite cells, fibroblasts, epidermal cells, blood cells, myoblasts, myocytes, vascular endothelial cells, hepatocytes, pericytes, embryonic stem cells, extraembryonic cells, mesenchymal stem cells, somatic cells, adipocytes, chondrocytes and/or fibroadipogenic progenitor cells. The heterogeneous cell population preferably comprises or consists of mononuclear cells.

Endogenous satellite cells are typically found sandwiched between the basement membrane and sarcolemma (cell membrane) of individual muscle fibres. In undamaged muscle, the majority of satellite cells are quiescent; they neither differentiate nor undergo cell division. But in response to mechanical stress, satellite cells can be activated. Satellite cells that are isolated from a muscle biopsy can also become activated during muscle removal and tissue digestion. Activated satellite cells can initially proliferate as myoblasts before undergoing further differentiation to form myocytes and/or myotubes. Myotubes are multi-nuclear cells that are formed from the fusion of multiple myoblasts and/or myocytes. Myotubes may further mature to form myofibres by expressing various cytoskeletal and structural proteins.

The heterogenous cell population may be obtained from an animal. For example, the heterogenous cell population may be obtained from a tissue sample taken from a live animal or an animal carcass. In some examples, the heterogeneous cell population is obtained from the skeletal muscle of an animal. In some examples, the heterogeneous cell population is obtained from embryonic tissue of an animal.

The heterogenous cell population may be obtained from livestock, such as bovine, porcine, sheep, goat, camel, llama, buffalo, rabbit etc. In some examples, the heterogenous cell population is obtained from poultry, such as chicken, turkey, duck, goose, quail, pheasant, pigeon etc. In some examples, the heterogenous cell population is obtained from game species such as deer, kangaroo, gallinaceous fowl, waterfowl, hare etc. In some examples, the heterogenous cell population is obtained from aquatic species or semi-aquatic species, such as fish, crustacean, mollusc, cephalopod, cetacean, crocodilian, turtle, frog etc. In some examples, the heterogenous cell population is obtained from exotic, conserved, endangered or extinct animal species. In some examples, the heterogenous cell population is obtained from a human, primate, rodent or companion animal, such as a dog, a cat or a horse.

Non-limiting examples of non-human animals from which the heterogeneous cell population may be obtained include alpaca, antelope, bear, beaver, bison, boar, camel, caribou, cattle, chicken, clam, crab, deer, duck, elephant, elk, emu, fish, fox, frog, giraffe, goat, grouse, hare, horse, ibex, kangaroo, koala, lamb, lion, lizard, llama, lobster, moose, mouse, mussel, octopus, ostrich, oyster, peccary, pheasant, pig, pigeon, prawn, quail, rabbit, rat, seal, shark, sheep, snake, squid, squirrel, tiger, turkey, turtle, whale, wombat, yak and zebra. In certain examples, the progenitor cells may be obtained from Bos taurus, Bos grunniens, Gallus gallus, Meleagris gallopavo, Sus scrofa, Anas platyrhynchos, Salmo salar, Thunnus thynnus, Ovis aries, Coturnix coturnix, Capra aegagrus, Homarus americanus, Mus musculus, Oreochromis sp., Mus musculus, Oncorhynus mykiss, Vicugna pacos, Lama glama, Camelus sp., Equine ferus, Equus sp., a Cervidae, a Leporidae, Dromaius novaehollandiae, or a Macropodidae such as Osphranter rufus, Macropus giganteous, Macropus fuliginosus or Osphranter antilopinus.

### Selective Culturing

The heterogeneous cell population is selectively cultured to produce a selected cell population. Selective culturing involves culturing cells under defined conditions that enable cells having a particular phenotype to be distinguished (and selectively propagated) from cells that do not have that phenotype. For example, the heterogeneous cell population may be cultured under heat stress such that heat-tolerant cells can be identified and separated from heat-intolerant cells, and then subcultured. In another example, the heterogeneous cell population may be selectively cultured under chemical stress (eg, nutrient limitation, high or low salt concentration etc.).

Preferably, the heterogeneous cell population is selectively cultured based on adherence. For example, the heterogeneous cell population may be cultured in a plastic vessel, such as in a well of a multi-well plate that does not comprise an adherence-enhancing substrate and then cells present in suspension (non-adherent) can be separated from cells that are adhered to the vessel (adherent). Non-adherent cells can be separated from adherent cells by transferring the growth medium, which will include the non-adherent cells, into a new vessel comprising an adherence-enhancing substrate. Alternatively, the heterogeneous cell population may be cultured in a plastic vessel which does comprise an adherence-enhancing subtrate, but for a shorter period of time. Again, non-adherent cells can be separated from adherent cells by transferring the growth medium, which will include the non-adherent cells, into a new vessel comprising an adherence-enhancing substrate. Any suitable vessel may be used to selectively culture cells, eg, a well of a multi-well plate, a beaker, a flask etc.

It will be understood that cells that are referred to as “non-adherent” may still be capable of adherent growth, but their capacity for adherent growth may be less than that of adherent cells, or they may be capable of adherent growth only in the presence of an adherence-enhancing substrate. The “non-adherent” cells may be thought of as weakly adherent while the “adherent” cells may be thought of as strongly adherent. It has been found that adherent (ie, strongly adherent) cells derived from skeletal muscle tissue predominantly comprise fibroblasts whereas non-adherent (ie, weakly adherent) cells predominantly comprise skeletal muscle stem cells. It has also been found that adherent cells derived from embryonic tissue predominantly comprise embryonic fibroblasts.

Adherence-enhancing substrates include substrates that promote adherent cell growth. Suitable adherence-enhancing substrates may include gelatin, collagen, laminin, matrigel or other extracellular-matrix derived coatings. Those skilled in the art will understand that other adherence-enhancing substrates could be suitably employed in these methods.

Cells that are adhered to the vessel or to the adherence-enhancing substrate may be cultured for 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks or longer. Preferably, the cells are cultured for up to 2 weeks. The cells may be cultured at between about 25° C. and 45° C., for example at between about 26° C. and 45° C., or between about 27° C. and 45° C., or between about 28° C. and 45° C., or between about 29° C. and 45° C., or between about 30° C. and 45° C., or between about 30° C. and 44° C., or between about 30° C. and 43° C., or between about 30° C. and 42° C. In some examples, the cells are obtained from a reptile and are cultured at about 30° C. In some examples, the cells are obtained from a mammal or marsupial and are cultured at about 37° C. In some examples, the cells are obtained from a bird and are cultured at about 42° C.

The cells may be cultured in any suitable growth medium, and those skilled in the art will be aware of many such media. For example, a suitable growth medium may comprise basal medium (DMEM/F12), fetal bovine serum and antibiotics such as penicillin and streptomycin. General classes of antibiotics that may be suitable for use in the growth medium include, for example, aminoglycosides, polyenes, nitroimidazole, rifamycins, bacitracin, beta-lactam antibiotics, cephalosporins, chloramphenicol, macrolides, lincosamides, penicillins, quinolones, rifampicin, glycopeptide, tetracyclines, trimethoprim and sulfonamides. Examples of suitable antibiotics for use in accordance with the present disclosure may include amoxicillin, augmentin, amoxicillin, ampicillin, azlocillin, flucloxacillin, mezlocillin, methicillin, penicillin G, penicillin V, cephalexin, cefazedone, cefuroxime, loracarbef, cemetazole, cefotetan, cefoxitin, ciprofloxacin, levaquin, and floxacin, tetracycline, doxycycline, or minocycline, gentamycin, amikacin, and tobramycin, clarithromycin, azithromycin, erythromycin, daptomycin, neomycin, kanamycin or streptomycin. Growth media for non-adherent cells may be supplemented with fibroblast growth factor 2 (FGF2). In some examples, the medium is substantially free of serum.

The selective culturing may comprise an initial phase of incubation followed a subculturing phase. Adherent cells are preferably subcultured in the presence of an adherence-enhancing substrate but may be subcultured in the absence of an adherence-enhancing substrate. Non-adherent cells may be subcultured in the presence of an adherence-enhancing substrate. The cells may be dissociated from their substrate (eg, from the vessel wall or the adherence-enhancing substrate) with, for example, a dissociation enzyme (eg, TrypLE or trypsin), an enzyme free dissociation buffer (eg, Cell Dissociation Buffer from Thermo), or by physical agitation. The cells can then be subcultured in fresh growth media and a new vessel. The cells may be resuspended in fresh growth media at a density of between about 2,000 and 10,000 cells/cm², or between about 2,500 and 9,000 cells/cm², or between about 3,000 and 8,000 cells/cm², or between about 3,500 and 7,500 cells/cm², or between about 4,000 and 7,000 cells/cm², or between about 4,500 and 7,000 cells/cm², or between about 5,000 and 7,000 cells/cm², or between about 5,500 and 7,000 cells/cm², or between about 5,500 and 6,500 cells/cm². This process of dissociation, resuspension and subculturing may be repeated 2, 3, 4, 5, 6 or more times. The cells may be dissociated after a certain number of days in culture, for example, after 1 day, 2 days, 3 days, 4 days, 5 days, 6 days, 1 week, 8 days, 9 days, 10 days, 11 days, 12 days, 13 days, or 2 weeks in culture, or once they cover more than a particular percentage of the surface of the substrate. For example, the cells may be dissociated once they cover more than about 10% of the surface of the substrate, or more than about 15%, or more than about 20%, or more than about 25%, or more than about 30%, or more than about 35%, or more than about 40%, or more than about 45%, or more than about 50%, or more than about 55%, or more than about 60%, or more than about 65%, or more than about 70%, or more than about 75%, or more than about 80%, or more than about 85%, or more than about 90% of the surface of the substrate. Preferably, the cells are dissociated once they cover between about 30% and 60% of the surface of the substrate.

In some examples, the present disclosure provides a method of producing an enriched cell population comprising:
selectively culturing a heterogenous cell population to produce a selected cell population, wherein the selective culturing comprises:
   incubating the heterogenous cell population in a growth medium and selecting cells having a defined phenotype, and
   subculturing the cells having a defined phenotype to produce the selected cell population;
sorting the selected cell population by FACS to produce a population of sorted cells; and
culturing the population of sorted cells to produce an enriched cell population.

In some examples, the present disclosure provides a method of producing an enriched cell population comprising:
selectively culturing a heterogenous cell population to produce a selected cell population, wherein the selective culturing comprises:
   incubating the heterogenous cell population in a growth medium and selecting cells having a defined phenotype, and
   iteratively subculturing the cells having a defined phenotype to produce the selected cell population;
sorting the selected cell population by FACS to produce a population of sorted cells; and
culturing the population of sorted cells to produce an enriched cell population.

The incubation phase may be performed for between about 30 minutes and 48 hours, or between about 30 minutes and 36 hours, or between about 30 minutes and 30 hours, or between about 45 minutes and 30 hours, or between about 1 hour and 30 hours, or between about 1 hour and 24 hours. The subculturing phase, which may include iterative subculturing, may be performed over a period of 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks or longer.

In some examples, the present disclosure provides a method of producing an enriched cell population comprising:
selectively culturing a heterogenous cell population to produce a selected cell population, wherein the selective culturing comprises:
   incubating the heterogenous cell population in a growth medium for between about 1 hour and 24 hours and selecting cells having a defined phenotype, and
   iteratively subculturing the cells having a defined phenotype over a period of about 3 to 5 weeks to produce the selected cell population;
sorting the selected cell population by FACS to produce a population of sorted cells; and
culturing the population of sorted cells to produce an enriched cell population.

It will be understood that subculturing involves transferring some cells from a population of cells to fresh growth media and futher culturing those cells. Iterative subculturing involves repeating this process 1, 2, 3, 4 or more times. In the context of selective culturing, each round of iterative subculture may include a defined condition that enables cells having a particular phenotype to be distinguished and selectively propagated from cells that do not have that phenotype.

### Cell Sorting

By selectively culturing the heterogeneous cell population, a selected cell population having a defined phenotype is obtained. The selected cell population can then be sorted to produce a population of sorted cells. The selected cell population may be sorted by fluorescence-activated cell sorting (FACS), magnetic-activated cell sorting (MACS) or by serial dilution. Preferably the cells are sorted by FACS.

The selected cell population can be sorted based on any characteristic, or combination of characteristics, of interest. For example, FACS may be used to sort cells based on their size, granularity and/or viability. The selected cell population may be stained with hoeschst and subsequently sorted based on viability. Preferably, the cell sorting does not require an antibody or involve antibody-based selection. In some examples, the present disclosure provides a method of producing an enriched cell population comprising:
selectively culturing a heterogenous cell population to produce a selected cell population;
sorting the selected cell population based on cell size and/or viability by FACS to produce a population of sorted cells;
culturing the sorted cells to produce an enriched cell population.

In some examples, the present disclosure provides a method of producing an enriched cell population comprising:
selectively culturing a heterogenous cell population to produce a selected cell population, wherein the selective culturing comprises:
   incubating the heterogenous cell population in a growth medium and selecting cells having a defined phenotype, and
   subculturing the cells having a defined phenotype to produce the selected cell population;
sorting the selected cell population to produce a population of sorted cells; and
culturing the population of sorted cells to produce an enriched cell population, wherein the step of culturing the population of sorted cells comprises:
   selecting sorted cells having a defined characteristic from the population of sorted cells; and
   subculturing the sorted cells having a defined characteristic to produce the enriched cell population.

In some examples, the present disclosure provides a method of producing an enriched cell population comprising:
selectively culturing a heterogenous cell population to produce a selected cell population, wherein the selective culturing comprises:
   incubating the heterogenous cell population in a growth medium and selecting cells having a defined phenotype, and
   subculturing the cells having a defined phenotype to produce the selected cell population;
sorting the selected cell population by FACS to produce a population of sorted cells, wherein the FACS does not require an antibody to sort the selected cell population; and
culturing the population of sorted cells to produce an enriched cell population, wherein the step of culturing the population of sorted cells comprises:
   selecting sorted cells having a defined characteristic from the population of sorted cells; and
   subculturing the sorted cells having a defined characteristic to produce the enriched cell population.

The selected cell population is preferably suspended in a FACS buffer (eg, comprising basal medium (DMEM/F12) and 2% fetal bovine serum) prior to cell sorting. The FACS buffer is preferably chilled.

The sorted cells are preferably distributed across a plurality of wells of a multi-well plate. The wells preferably contain a suitable growth medium, and may also contain an adherence-enhancing substrate such as gelatin. The sorted cells may, for example, be distributed across the wells of multiple 384-well plates.

The multi-well plate(s) containing the sorted cells may be imaged at specific intervals over a period of hours, days or weeks to identify wells containing cells having a defined characteristic. For example, the defined characteristic may be growth rate, and cells having that characteristic may be identified as being those present in high abundance within a well relative to other wells of the multi-well plate. For example, the top 25% of wells (96 wells of a 384-well plate) having cells in the highest abundance relative to the other 75% of wells may be considered to contain cells having a high growth rate. In some examples, the top 20% of wells, or top 15% of wells, or top 10% of wells, or top 5% of wells or top 1% of wells having cells in the highest abundance may be considered to contain cells having a high growth rate. Sorted cells identified as having a defined characteristic may be dissociated from the multi-well plate, transferred to a new multi-plate containing fresh growth medium and subcultured. For example, the sorted cells having a defined characteristic may be transferred from a 384-well plate to a 96-well plate. The cells may then be cultured and imaged to again identify cells having a defined characteristic, for example, high abundance within a well relative to other wells of the 96-well plate. The cells identified as having a defined characteristic may again be selected, transferred to a 24-well plate and subcultured. This process of selection and subculturing can be performed iteratively until an enriched cell population having a desired purity is obtained. It will be understood that cell purity here refers to the concentration of a specific cell type in the enriched cell population relative to other cell types. For example, an enriched cell population may be 100% pure in the sense that it consists of a single cell type. It has been found that the enriched cell population produced by the methods of the present disclosure may be derived from a single cell line and is therefore 100% pure.

The population of sorted cells are cultured to produce an enriched cell population. The sorted cells may be cultured for at least 1 week, at least 2 weeks, at least 15 days, at least 16 days, at least 17 days, at least 18 days, at least 19 days, at least 20 days, at least 3 weeks, at least 22 days, at least 23 days, at least 24 days, at least 25 days, at least 26 days, at least 27 days, at least 4 weeks, at least 29 days, at least 30 days, at least 31 days, at least 32 days, at least 33 days, at least 34 days, at least 5 weeks, at least 36 days, at least 37 days, at least 38 days, at least 39 days, at least 40 days, at least 41 days or at least 6 weeks. This may include a period in which the sorted cells are cultured immediately following FACS followed by a period in which the cells are iteratively selected and subcultured. For example, the sorted cells may be cultured for at least 1 day, at least 2 days, at least 3 days, at least 4 days, at least 5 days, at least 6 days, at least 1 week, at least 2 weeks, at least 15 days, at least 16 days, at least 17 days, at least 18 days, at least 19 days, at least 20 days, or at least 3 weeks, followed by a period of iterative selection and subculturing for at least a further 5 days, 6 days, 1 week, 8 days, 9 days, 10 days, 11 days, 12 days, 13 days, 2 weeks, 15 days, 16 days, 17 days, 18 days, 19 days, 20 days, or 3 weeks.

In some examples, the present disclosure provides a method of producing an enriched cell population comprising:
selectively culturing a heterogenous cell population to produce a selected cell population;
sorting the selected cell population by FACS to produce a population of sorted cells;
selecting sorted cells having a high growth rate; and
subculturing the sorted cells having a high growth rate to produce an enriched cell population.

In some examples, the present disclosure provides a method of producing an enriched cell population comprising:
selectively culturing a heterogenous cell population to produce a selected cell population;
sorting the selected cell population by FACS to produce a population of sorted cells;
selecting sorted cells having a high growth rate by culturing the population of sorted cells and selecting those that replicate faster relative to other sorted cells from the population of sorted cells; and
subculturing the sorted cells having a high growth rate to produce an enriched cell population.

In some examples, the present disclosure provides a method of producing an enriched cell population comprising:
selectively culturing a heterogenous cell population to produce a selected cell population;
sorting the selected cell population by FACS to produce a population of sorted cells;
selecting sorted cells having a high growth rate by culturing the population of sorted cells and selecting those that replicate faster relative to other sorted cells from the population of sorted cells; and
iteratively subculturing the sorted cells having a high growth rate to produce an enriched cell population.

### Enriched Cell Population

The enriched cell population may be enriched for muscle cells, fibroblasts, myocytes, adipocytes, endothelial cells, epidermal cells, fibroadipogenic progenitor cells and/or myoblasts.

The methods described herein may enrich for a particular cell type present in the heterogenous cell population by at least 2-fold, 3-fold, 4-fold, 5-fold, 6-fold, 7-fold, 8-fold, 9-fold or 10-fold. For example, a cell type that constitutes 10% of the heterogeneous cell population may constitute 20% of the enriched cell population. Preferably, the methods described herein produce an enriched cell population in which more that 90% of cells are a single cell type. Preferably, at least 95% of cells in the enriched cell population are a single cell type. Preferably, at least 96% of cells in the enriched cell population are a single cell type. Preferably, at least 97% of cells in the enriched cell population are a single cell type. Preferably, at least 98% of cells in the enriched cell population are a single cell type. Preferably, at least 99% of cells in the enriched cell population are a single cell type. Preferably, the enriched cell population consists of a single cell type. In some examples, the enriched cell population consists of no more than 5 cell types, or no more than 4 cell types, or no more than 3 cell types, or no more than 2 cell types. In some examples, at least 95% of cells in the enriched cell population consist of no more than 5 cell types, or no more than 4 cell types, or no more than 3 cell types, or no more than 2 cell types. In some examples, at least 96% of cells in the enriched cell population consist of no more than 5 cell types, or no more than 4 cell types, or no more than 3 cell types, or no more than 2 cell types. In some examples, at least 97% of cells in the enriched cell population consist of no more than 5 cell types, or no more than 4 cell types, or no more than 3 cell types, or no more than 2 cell types. In some examples, at least 98% of cells in the enriched cell population consist of no more than 5 cell types, or no more than 4 cell types, or no more than 3 cell types, or no more than 2 cell types. In some examples, at least 99% of cells in the enriched cell population consist of no more than 5 cell types, or no more than 4 cell types, or no more than 3 cell types, or no more than 2 cell types. Preferably, the enriched cell population is derived from a single cell line. Preferably, the enriched cell population is enriched with myoblasts or fibroblasts.

In some examples, the present disclosure provides a method of producing an enriched fibroblast population, the method comprising:
selectively culturing a heterogenous cell population obtained from skeletal muscle of an animal to produce a selected cell population, wherein the selective culturing comprises:
   incubating the heterogenous cell population in a vessel that does not comprise an adherence-enhancing substrate and selecting adherent cells, and
   subculturing the adherent cells in a vessel that does not comprise an adherence-enhancing substrate to produce the selected cell population;
sorting the selected cell population by FACS to produce a population of sorted cells; and
culturing the population of sorted cells to produce an enriched fibroblast population.

In some examples, the present disclosure provides a method of producing an enriched fibroblast population, the method comprising:
selectively culturing a heterogenous cell population obtained from embryonic tissue of an animal to produce a selected cell population, wherein the selective culturing comprises:
   incubating the heterogenous cell population in a vessel that does not comprise an adherence-enhancing substrate and selecting adherent cells, and
   subculturing the adherent cells in a vessel that does not comprise an adherence-enhancing substrate to produce the selected cell population;
sorting the selected cell population by FACS to produce a population of sorted cells; and
culturing the population of sorted cells to produce an enriched embryonic fibroblast population.

In some examples, the present disclosure provides a method of producing an enriched myoblast population, the method comprising:
selectively culturing a heterogenous cell population obtained from embryonic tissue of an animal to produce a selected cell population, wherein the selective culturing comprises:
   incubating the heterogenous cell population in a vessel that does not comprise an adherence-enhancing substrate and selecting nonadherent cells,
   subculturing the nonadherent cells in a vessel that comprises an adherence-enhancing substrate to produce the selected cell population;
sorting the selected cell population by FACS to produce a population of sorted cells; and
culturing the population of sorted cells to produce an enriched cell population.

In some examples, the present disclosure provides a method of producing an enriched myoblast population, the method comprising:
selectively culturing a heterogenous cell population obtained from skeletal muscle of an animal to produce a selected cell population, wherein the selective culturing comprises:
   incubating the heterogenous cell population in a vessel that does not comprise an adherence-enhancing substrate and selecting nonadherent cells,
   subculturing the nonadherent cells in a vessel that comprises an adherence-enhancing substrate to produce the selected cell population;
sorting the selected cell population by FACS to produce a population of sorted cells; and
culturing the population of sorted cells to produce an enriched myoblast population.

### Food Compositions

Muscle cells produced according to the present disclosure may be processed into an edible product such as a human food composition or a non-human animal food composition or both. In some examples, the muscle cells are intended for human consumption. In other examples, the muscle cells are to be used in animal feed such as feed for livestock, feed for aquaculture or feed for companion animals. The muscle cells may be processed into a raw, uncooked edible product, a cooked edible product or a cooked or uncooked food ingredient. Processing may comprise exposing the cellular biomass to sub-physiological temperatures that do not support expansion and/or differentiation of the cells.

Those skilled in the art will appreciate that a food composition may be selected according to physical properties (eg, Young's modulus, viscosity modulus, stiffness etc.) and a desired use (eg, consumption by a human adult). The food composition may comprise ingredients that provide a desired texture, moisture retention, product adhesion, flavour, nutritional property or colour. In some examples, the food composition comprises a binder or extender. Examples of binders include starch, such as potato starch, flour, egg, gelatin, carrageenan and tapioca flour. Examples of extenders include soy protein, milk protein and meat-derived protein. Extracellular matrix proteins may be added to modulate texture. The food composition may be supplemented with one or more fatty acids, such as a polyunsaturated fatty acid or a monounsaturated fatty acid. The fatty acid may be an omega 3 fatty acid. In some examples, the food composition may be supplemented with palmitic acid, oleic acid, docosahexaenoic acid, stearic acid, linoleic acid, linolenic acid, arachidonic acid or eicosapentaenoic acid.

The food composition may comprise other ingredients that contribute to the flavour, smell, texture, nutritional quality, colour, shelf-life, storability, organoleptic properties, texture etc. Additional ingredients may include a fat, sodium, potassium, monosodium glutamate, cholesterol, sodium chloride, iodine, starch, a sugar such as sucrose, glucose or fructose, oil such as vegetable oil, canola oil, peanut oil, coconut oil or sesame oil, grains, flour, vitamins, minerals, amino acids, zinc, selenium, fibre, spices, soybean extract, an emulsifier, a humectant, an acidity regulator, an anticaking agent, a preservative, a sweetener or a thickener.

### Therapeutic Applications

While the cells produced according to the present disclosure may be used to produce food compositions, it will be appreciated that the cells may also have therapeutic applications. For example, muscle cells produced according to the present disclosure may be useful in the treatment of a skeletal muscle-associated disease or disorder. In that regard, the present disclosure provides a method of treating a muscle-associated disease or disorder in a subject the method comprising administering to the subject muscle cells produced according to the present disclosure.

Skeletal muscle disorders may be primary disorders of muscle energy metabolism, including defects in muscle carbohydrate and lipid metabolism, disorders of mitochondrial electron transport and abnormalities of purine nucleotide metabolism affecting the capacity for ATP resynthesis. For example, oxidative phosphorylation is the dominant quantitative source of energy for ATP resynthesis under most exercise conditions. As such, disordered oxidative metabolism (eg, in patients with defects in the availability or utilisation of oxidisable substrates, such as those with phosphorylase or PFK deficiency or those with defects in mitochondrial electron transport) may lead to severely impaired skeletal muscle performance, intolerance to sustained exercise or premature fatigue. Another group of disorders includes patients with decreased muscle mass due to muscle necrosis, atrophy or replacement of muscle by fat and connective tissue. These disorders are exemplified by the various muscular dystrophies (eg, Duchenne's dystrophy, Becker's dystrophy, LG dystrophy, FSH dystrophy and myotonic dystrophy) in which skeletal muscle performance is severely impaired due to muscle wasting and weakness notwithstanding largely normal pathways for muscle ATP resynthesis.

Other relevant muscle disorders may include muscle atrophy, myotonia, sarcopenia, cachexia, a congenital myopathy, a mitochondrial myopathy, an inflammatory myopathy, a metabolic myopathy and a muscular dystrophy such as Duchenne's dystrophy, Becker's dystrophy, Corneal dystrophy, Conal dystrophy, Retinal dystrophy, Reflex sympathetic dystrophy, LG dystrophy, FSH dystrophy or myotonic dystrophy. In some examples, the muscle cells may be autologous, ie, they may be derived from the subject who is to be treated.

The muscle cells produced by the methods of the present disclosure may also be suitable for disease modelling. For example, the consequence of genetic defects or external stimuli on muscle development or function may be investigated, such as by determining the contractile properties of muscle cells carrying those genetic defects or exposed to those external stimuli. In further examples, the efficacy of drugs or other molecules in treating the genetic defect may be assessed using muscle cells produced in accordance with the present disclosure.

In some examples, muscle cells produced according to the present disclosure may be useful for toxicity screening or in vitro drug testing. Some drugs used for therapeutic interventions may cause unexpected toxicity in muscle tissue, possibly leading to significant morbidity and disability. Myotoxic drugs can cause myopathies through a range of mechanisms, including by directly affecting muscle organelles and other subcellular structures such as mitochondria, lysosomes and myofibrillar proteins; altering muscle antigens and generating an immunologic or inflammatory reaction; or by disturbing the electrolyte or nutritional balance, which can subsequently affect muscle function. Muscle tissue can be particularly susceptible to drug-related injury due to its mass, high blood flow and energy metabolism. In that regard, drugs may be screened against muscle cells produced according to the present disclosure to determine general toxicity or to determine whether or not muscle cells from a particular individual display sensitivity to drugs or other molecules.

In some examples, muscle cells produced according to the present disclosure may be derived from a subject having one or more genetic defects. The present disclosure contemplates a genetic background test wherein a candidate drug, molecule or other stimulus is tested against muscle cells carrying a particular genetic defect to determine whether there are differential efficacies and/or side effects that correlate with a particular genetic background. This may enable selection of specific therapies for patients with a particular genetic background.

### Items of the Present Disclosure

Set forth below are non-limiting methods and compositions of the present disclosure.

Clause 1. A method of producing an enriched cell population, the method comprising:
selectively culturing a heterogenous cell population to produce a selected cell population;
sorting the selected cell population to produce a population of sorted cells; and
culturing the population of sorted cells to produce an enriched cell population.

Clause 2. The method of clause 1 wherein the selective culturing comprises culturing cells of the heterogeneous cell population and selecting cells having a defined phenotype.

Clause 3. The method of clause 2 wherein the defined phenotype is adherence.

Clause 4. The method of any one of clauses 1 to 3 wherein the selective culturing comprises culturing cells of the heterogeneous cell population in a vessel that does not comprise an adherence-enhancing substrate and selecting adherent cells.

Clause 5. The method of any one of clauses 1 to 3 wherein the selective culturing comprises culturing cells of the heterogeneous cell population in a vessel that does not comprise an adherence-enhancing substrate and selecting non-adherent cells.

Clause 6. The method of clauses 5 wherein the selective culturing further comprises subculturing the non-adherent cells in a vessel comprising an adherence-enhancing substrate.

Clause 7. The method of any one of clauses 4 to 6 wherein the adherence-enhancing substrate is gelatin.

Clause 8. The method of any one of clauses 1 to 7 wherein the selective culturing is performed for at least two weeks.

Clause 9. The method of any one of clauses 1 to 8 wherein the selective culturing comprises dissociating cells from a substrate and subculturing in fresh growth medium.

Clause 10. The method of clause 9 wherein the selective culturing comprises iteratively dissociating cells from a substrate and subculturing in fresh growth medium two or more times.

Clause 11. The method of any one of clauses 1 to 10 wherein the selected cell population is sorted by fluorescence-activated cell sorting (FACS).

Clause 12. The method of clause 11 wherein the FACS sorts the selected cell population based on cell size.

Clause 13. The method of clause 11 or clause 12 wherein the FACS sorts the selected cell population based on cell viability.

Clause 14. The method of any one of clauses 11 to 13 wherein the FACS does not require an antibody to sort the selected cell population.

Clause 15. The method of any one of clauses 1 to 14 wherein sorted cells having a defined characteristic are selected and subcultured to produce the enriched cell population.

Clause 16. The method of any one of clauses 1 to 15 wherein the step of culturing the population of sorted cells comprises:
selecting sorted cells having a defined characteristic from the population of sorted cells; and
subculturing the sorted cells having a defined characteristic to produce the enriched cell population.

Clause 17. The method of clause 15 or clause 16 wherein the defined characteristic is growth rate or abundance.

Clause 18. The method of any one of clauses 1 to 17 wherein the population of sorted cells is distributed across a plurality of wells of a multi-well plate.

Clause 19. The method of clause 18 wherein the wells are imaged and sorted cells having a defined characteristic are selected and subcultured to produce the enriched cell population.

Clause 20. The method of any one of clauses 15 to 19 wherein the sorted cells having a defined characteristic are iteratively selected and subcultured.

Clause 21. The method of any one of clauses 1 to 20 wherein the heterogenous cell population is obtained from an animal.

Clause 22. The method of clause 21 wherein the animal is a mammal, a marsupial, a reptile or a bird.

Clause 23. The method of any one of clauses 1 to 22 wherein the enriched cell population consists of less than four cell types.

Clause 24. The method of any one of clauses 1 to 23 wherein the enriched cell population consists of one cell type.

Clause 25. The method of any one of clauses 1 to 23 wherein at least 98% of cells in the enriched cell population consist of one cell type.

Clause 26. The method of any one of clauses 23 to 25 wherein the cell type is a myoblast or a fibroblast.

Clause 27. A method of producing an enriched cell population, the method comprising:
sorting a selected cell population to produce a population of sorted cells; and
culturing the population of sorted cells to produce an enriched cell population,
wherein the selected cell population is obtained by selectively culturing a heterogenous cell population to produce a selected cell population.

Clause 28. An enriched cell population produced by the method of any one of clauses 1 to 27.

Clause 29. A food composition comprising an enriched cell population produced by the method of any one of clauses 1 to 27.

### EXAMPLES

### Tissue Preparation

### Skeletal Muscle Tissue

A tissue sample of 3 to 5 grams was harvested from the skeletal muscle of an animal [a mammal (sheep), a marsupial (Bennets Wallaby), a reptile (saltwater crocodile) and a bird (chicken)] within 48 hrs post-mortem. The skin of the animal was sprayed with 70% ethanol and then washed with an iodine solution. A small square of the skin (about 7×7 cm) was removed using a scalpel and tweezers. A fresh set of sterile scalpel and tweezers was then used to remove the tissue sample; a piece of muscle about 3×2×2 cm.

Tissue samples were briefly washed in phosphate buffered saline containing penicillin and streptomycin, and then quickly blotted to remove any blood. At this stage any adherent fat or connective tissue was removed (using sterile forceps and surgical scissors) before the tissue was transferred to a tissue culture plate containing digestion buffer I [comprising: a basal media (DMEM/F12), 10% horse serum, 1× penicillin and streptomycin, and 750 U/ml collagenase (type II)].

The tissue sample was thoroughly minced using two sterile scalpel blades.

The minced muscle sample in digestion buffer was then transferred to a sealed container and incubated for 1 hour in a shaking water bath set to 37° C. for mammal and marsupial tissue samples, 42° C. for avian tissue sampes and 30° C. for reptile tissue samples.

After the 1-hour incubation period, the sample was centrifuged to pellet all cells and tissue debris, and the digestion buffer was aspirated. Digestion buffer II (comprising: a basal media (DMEM/F12), horse serum, penicillin and streptomycin, collagenase (type II) and dispase) was then added to the cell pellet and was incubated in a shaking water bath set to 37° C. (mammal and marsupial), 42° C. (avian) or 30° C. (reptile) for 1 hour.

The cell mix was triturated several times through a 10 mL serological pipette and then passed up and down through a 20-gauge needle attached to a 10 ml syringe. The triturated cell mix was then centrifuged to pellet the cells and the digestion buffer was replaced with wash media [comprising: a basal media (DMEM/F12), horse serum, penicillin and streptomycin] and the cells resuspended.

The wash media containing the resuspended cells was passed through a 40 μm filter to remove cellular debris and collected in a 50 mL tube. An additional volume of wash media (5-10 mL) was passed through the filter to ensure all cells were collected.

Each tube containing cells was filled to 50 mL with wash media and then centrifuged to pellet the cells.

All wash media was then aspirated and replaced with 3 mL of pre-warmed (37° C. (mammal and marsupial), 42° C. (avian) or 30° C. (reptile)) growth media (comprising: a basal media (DMEM/F12), fetal bovine serum, penicillin and streptomycin).

The heterogenous cell population was resuspended using gentle trituration and then seeded into one well of an uncoated 6-well plate (FIG. 5A).

The 6-well plate was then kept in a humidified incubator at normoxia, supplemented with 5% CO₂ and maintained at 37° C. (mammal and marsupial), 42° C. (avian) or 30° C. (reptile).

### Embryonic Tissue

Freshly laid (fertilized) eggs were collected from duck, peacock, chicken, quail, turkey and pigeon within 24 hours of being laid. Fertile eggs were kept at room temperature (14-18 C) for between 24 hours and 1 week prior to commencing incubation.

Eggs were placed in incubators with aircells orientating upwards. Eggs were incubated in an Ova-Easy 100 egg dance series II incubator, a Flyline 36 egg incubator, or a Brinsea 14 egg Maxi II EX incubator and maintained at 35.5-37.5 C and 55-65% humidity. Eggs were rotated by 180° every 120 mins (with alternating rotational direction).

To confirm developmental progression, eggs were ‘candled’ at ¼ of the natural incubation period for the species, as well as immediately prior to termination. Eggs were removed from the incubator (eggs were not left out of the incubator for >10 min during candling). In a dark room, eggs were placed on a Titan Ultra Bright Egg Candler and gently rotated to observe features. Embryos were discarded during candling under the following conditions:
Cracked shells (source of possible contamination)
No visible vascularisation by ¼ the species natural incubation period (infertile)
Signs embryonic death (eg. blood rings, lack of separation between yolk and albumin, greatly enlarged air cells).

After completion of candling the eggs were returned to the incubator, again with the aircell pointing upwards.

Eggs were removed from the incubator at stage 30-35 of development (after the appearance of the beak but before the appearance of feathers). Inside a sterile biological safety cabinet each egg was cracked by gently tapping against the side of a petri dish, with the embryo transferred to the dish. The head was removed using sterilised stainless steel forceps and the body transferred to a 50 ml tube containing 2 mL of sterile phosphate buffered saline (PBS) for every cm² of tissue isolated. Embryonic cells were released from the embryo by gentle trituration with a serological pipette for 3-5 mins.

The resulting cell suspension was passed through a 70 μm cell strainer and then centrifuged at 500 g for 5 mins. The PBS was carefully removed so as to not disturb the cell pellet and the heterogeneous cell population was resuspended in growth media (made up of DMEM/F12+10% FBS or OptiPro+4 mM Glutamax) by gentle trituration. Cells of th heterogeneous cell population were seeded into uncoated T175-flasks (1× T75 flask per cm² of tissue isolated) and then carefully placed into a humidified incubator (5% CO₂ and 38.5° C.).

### Selective Culturing

The heterogenous cell population was cultured for between 1 and 24 hrs, and then the supernatant was transferred to a fresh well of a 6-well plate coated with a gelatin matrix to encourage cell adhesion. The original well was fed fresh growth media to support adhered (strongly adherent) cells and both plates were then returned to the humidified incubator. This step allows for the separation of strongly and weakly adherent cell types (FIG. 5B,C; FIG. 6B,C). When skeletal muscle tissue was used to obtain the heterogeneous cell population, it was found that the strongly adherent fraction of cells contains predominantly fibroblasts, while the weakly adherent fraction contains skeletal muscle stem cells. When embryonic tissue was used to obtain the heterogeneous cell population, it was found that the strongly adherent fraction of cells contains predominantly embryonic fibroblasts.

Both strongly and weakly adherent cell types were grown in culture for up to 2 weeks to increase cell number (FIG. 5D; FIG. 6D). Half of the volume of growth media was replaced every 48 hrs with fresh (prewarmed) growth media. After the first 24 hrs, growth media for weakly adherent cells was supplemented with 10 ng/ml of FGF2.

Once 30 to 60% of the well surface was covered in cells, the cells were released from the well via treatment with a dissociation enzyme (TrypLE).

Dissociated cells were then resuspended in fresh growth media and seeded into a larger vessel at a density of 5,500-6,500 cells/cm² (T25 flask).

Both strongly and weakly adherent cells were grown in culture for up to an additional week to further increase cell number. Half of the volume of growth media was replaced every 48 hrs with fresh (prewarmed) growth media.

Once 30 to 60% of the well surface was covered in cells, the cells were released from the well via treatment with a dissociation enzyme.

Dissociated cells were then resuspended in fresh growth media and seeded into a larger vessel at a density of 5,500-6,500 cells/cm² (T75 flask).

Both strongly and weakly adherent cells were grown in culture for up to an additional week to further increase cell number. Half of the volume of growth media was replaced every 48 hrs with fresh (prewarmed) growth media.

Once 30 to 60% of the well surface was covered in cells, the cells were released from the well via treatment with a dissociation enzyme.

Dissociated cells were then resuspended in fresh growth media and seeded into a larger vessel at a density of 5,500-6,500 cells/cm² (T175 flask).

### Cell Sorting

Following the selective culturing described above, about 5 to 20 million cells were obtained per T175 flask.

Up to 20× 384-well plates were coated with 1% gelatin solution (diluted in ultra pure (milli-Q) water) and then filled with 50 μL of fresh growth media (comprising: a basal media (DMEM/F12), fetal bovine serum, penicillin and streptomycin). Half the plates were supplemented with 10 ng/ml of FGF2 to support the growth of weakly adherent cells. Gelatin was not used (although it can be) when the selected cell population was produced by selecting for strongly adherent cells.

All plates were then stored in a humidified CO₂ incubator ready for cell sorting.

In preparation for cell sorting, cells were released from the flask via treatment with a dissociation enzyme (TrypLE) and collected into 3 to 5 mL of ice-cold FACS buffer (comprising: a basal media (DMEM/F12) and 2% fetal bovine serum).

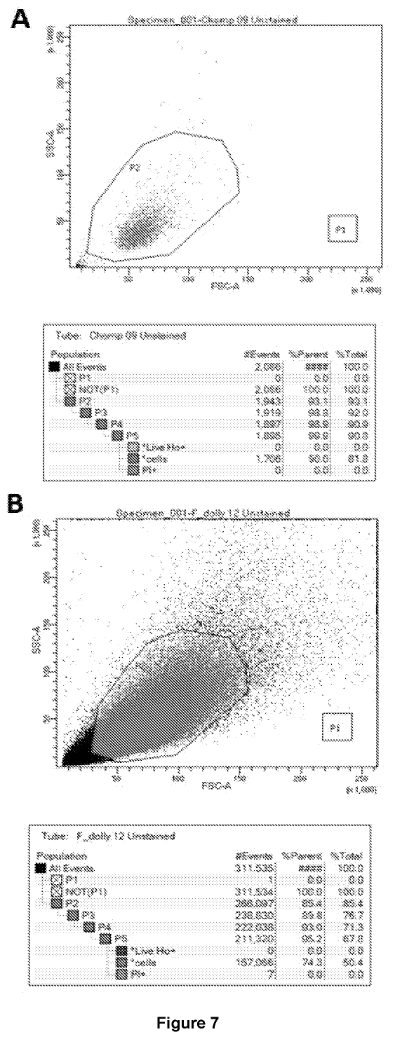

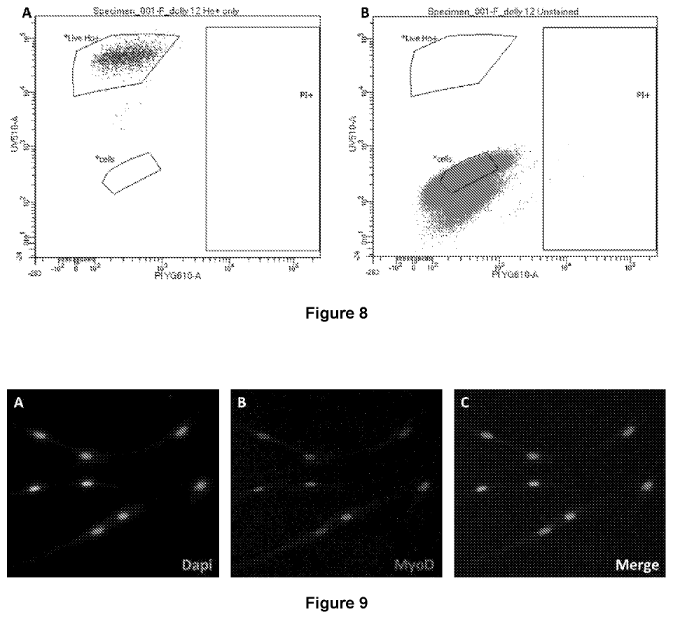

Immediately prior to sorting, cells were stained with hoechst 33342 (10 μg/mL) for 10 mins, followed by 2× 5 min washes in FACS buffer. In another experiment, unstained cells were sorted, and satisfactory results were also obtained (see FIGS. 7 and 8).

Single cells were sorted based on size, granularity and positive hoechst staining of the DNA into each well of a 384-well plate containing growth media using a FACSAriaIII equipped with a 100 μm-nozzle at 18 psi.

Immediately following sorting, each 384-well plate was returned to the humidified CO₂ incubator.

Every well of each 384-well plate was imaged on days 1, 2, 3, 6, 7, 10 and 13 using phase imaging. On day 15, the cells were stained with hoechst 33342 and imaged using a microscope equipped with a UV light source and filter.

Images from day 15 were analysed for total cell counts and the top 20% of wells containing the most cells were selected. The sorted cells in the 384-well plate may also be analysed for a defined characteristic such as growth rate using an automated imaging platform (e.g., Zaber) optionally combined with a machine learning image analysis algorithm. For example, at specific time points, each 384-well plate is loaded onto a mechanical stage mounted on a Zaber microscope. The mechanical stage automatically moves the 384-well plate and allows the sequential imaging of each well. Four images are taken of each well and then stitched together to cover the entire well, thus a total of 1,536 individual images (384 images after stitching) are obtained for each plate. Each image is then analysed, for example, using an algorithm trained to count adherent cells. The results allow the selection of the top 1% of wells containing the greatest number of cells.

Selected wells were washed with PBS and then released from the well via treatment with a dissociation enzyme.

Dissociated cells were then resuspended in fresh growth media and seeded into each well of a 96-well plate.

Every well of the 96-well plates was imaged on day 2. On day 5, the cells were stained with hoechst 33342 (10 μg/ml) and imaged using a microscope equipped with a UV light source and filter.

Images from day 5 were analysed for total cell counts and the top 20% of wells containing the most cells were selected.

Selected wells were washed with PBS and then released from the well via treatment with a dissociation enzyme.

Dissociated cells were then resuspended in fresh growth media and seeded into each well of a 24-well plate.

Every well from the 24-well plates was imaged on day 2. On day 5, the cells were stained with hoechst 33342 (10 μg/mL) and imaged using a microscope equipped with a UV light source and filter.

Images from day 5 were analysed for total cell counts and the top 20% of wells containing the most cells were selected.

Selected wells were washed with PBS and then released from the well via treatment with a dissociation enzyme.

Dissociated cells were then resuspended in fresh growth media and seeded into each well of a 6-well plate.

Every well from the 6-well plates was imaged on day 2. On day 5, the cells were stained with hoechst 33342 (10 μg/mL) and imaged using a microscope equipped with a UV light source and filter.

Images from day 5 were analysed for total cell counts and the top 20% of wells containing the most cells were selected.

Selected wells were washed with PBS and then released from the well via treatment with a dissociation enzyme.

A small aliquot of cells was taken for RNA isolation and immunofluorescence characterisation. To confirm the cell type of each cell line, immunofluorescence was perform using well-established markers for myoblasts (Pax7 and MyoD) and fibroblasts (vimenten and TE-7). FIG. 9 demonstrates pure sheep myoblast cells produced according to the method of the present disclosure.

All remaining cells were then resuspended in fresh growth media and seeded into a T25 flask for further expansion. At this stage, each cell line is derived from a single cell. Once 30-60% of the flask surface was covered in cells, the cells were released from the well via treatment with a dissociation enzyme.

Cells were pelleted via centrifugation, washed in ice-cold PBS and then stored in cryoprotectant (FBS+DMSO or cryostor) in liquid nitrogen.

It will be appreciated by those skilled in the art that the present disclosure may be embodied in many other forms.

1. A method of producing an enriched cell population, the method comprising:
selectively culturing a heterogenous cell population to produce a selected cell population;
sorting the selected cell population to produce a population of sorted cells; and
culturing the population of sorted cells to produce an enriched cell population.

2. The method of claim 1 wherein the selective culturing comprises culturing cells of the heterogeneous cell population and selecting cells having a defined phenotype.

3. The method of claim 2 wherein the defined phenotype is adherence.

4. The method of any one of claims 1 to 3 wherein the selective culturing comprises culturing cells of the heterogeneous cell population in a vessel that does not comprise an adherence-enhancing substrate and selecting adherent cells.

5. The method of any one of claims 1 to 3 wherein the selective culturing comprises culturing cells of the heterogeneous cell population in a vessel that does not comprise an adherence-enhancing substrate and selecting non-adherent cells.

6. The method of claim 5 wherein the selective culturing further comprises subculturing the non-adherent cells in a vessel comprising an adherence-enhancing substrate.

7. The method of any one of claims 4 to 6 wherein the adherence-enhancing substrate is gelatin.

8. The method of any one of claims 1 to 7 wherein the selective culturing is performed for at least two weeks.

9. The method of any one of claims 1 to 8 wherein the selective culturing comprises dissociating cells from a substrate and subculturing in fresh growth medium.

10. The method of claim 9 wherein the selective culturing comprises iteratively dissociating cells from a substrate and subculturing in fresh growth medium two or more times.

11. The method of any one of claims 1 to 10 wherein the selected cell population is sorted by fluorescence-activated cell sorting (FACS).

12. The method of claim 11 wherein the FACS sorts the selected cell population based on cell size.

13. The method of claim 11 or claim 12 wherein the FACS sorts the selected cell population based on cell viability.

14. The method of any one of claims 11 to 13 wherein the FACS does not require an antibody to sort the selected cell population.

15. The method of any one of claims 1 to 14 wherein sorted cells having a defined characteristic are selected and subcultured to produce the enriched cell population.

16. The method of any one of claims 1 to 15 wherein the step of culturing the population of sorted cells comprises:
selecting sorted cells having a defined characteristic from the population of sorted cells; and
subculturing the sorted cells having a defined characteristic to produce the enriched cell population.

17. The method of claim 15 or claim 16 wherein the defined characteristic is growth rate or abundance.

18. The method of any one of claims 1 to 17 wherein the population of sorted cells is distributed across a plurality of wells of a multi-well plate.

19. The method of claim 18 wherein the wells are imaged and sorted cells having a defined characteristic are selected and subcultured to produce the enriched cell population.

20. The method of any one of claims 15 to 19 wherein the sorted cells having a defined characteristic are iteratively selected and subcultured.

21. The method of any one of claims 1 to 20 wherein the heterogenous cell population is obtained from an animal.

22. The method of claim 21 wherein the animal is a mammal, a marsupial, a reptile or a bird.

23. The method of any one of claims 1 to 22 wherein the enriched cell population consists of less than four cell types.

24. The method of any one of claims 1 to 23 wherein the enriched cell population consists of one cell type.

25. The method of any one of claims 1 to 23 wherein at least 98% of cells in the enriched cell population consist of one cell type.

26. The method of any one of claims 23 to 25 wherein the cell type is a myoblast or a fibroblast.

27. A method of producing an enriched cell population, the method comprising:
sorting a selected cell population to produce a population of sorted cells; and
culturing the population of sorted cells to produce an enriched cell population,
wherein the selected cell population is obtained by selectively culturing a heterogenous cell population to produce a selected cell population.

28. An enriched cell population produced by the method of any one of claims 1 to 27.

29. A food composition comprising an enriched cell population produced by the method of any one of claims 1 to 27.
